(19) United States
(12) Patent Application Publication
(10) Pub. No.: US 2013/0142796 A1
(43) Pub. Date: Jun. 6, 2013

(54) TREATMENT FOR ANGIOGENIC DISORDERS

(71) Applicant: Subhransu RAY, Moraga, CA (US)

(72) Inventor: Subhransu RAY, Moraga, CA (US)

(21) Appl. No.: 13/705,983

(22) Filed: Dec. 5, 2012

Related U.S. Application Data
(60) Provisional application No. 61/567,051, filed on Dec. 5, 2011.

(51) Int. Cl. A61K 39/395; A61K 31/7052

(52) U.S. Cl. CPC A61K 39/3955; A61K 31/7052
USPC 424/134.1; 424/133.1

(57) ABSTRACT

Disclosed are pharmaceutical compositions comprising a therapeutically effective amount of a first compound, wherein the first compound binds the heparin-binding domain of the vascular endothelial growth factor (VEGF); and a therapeutically effective amount of a second compound, wherein the second compound binds to VEGF, thereby inhibiting the binding of VEGF to its cognate receptor. Also disclosed are methods of treating a VEGF-related disorder in a subject, the method comprising identifying a subject in need thereof, and administering to the subject a therapeutically effective amount of a first compound, wherein the first compound binds the heparin-binding domain of the vascular endothelial growth factor (VEGF); and a therapeutically effective amount of a second compound, wherein the second compound binds to VEGF, thereby inhibiting the binding of VEGF to its cognate receptor.

### RELATED APPLICATIONS

The present application claims priority to the U.S. Provisional Application Ser. No. 61/567,051, filed on Dec. 5, 2011 by Subhransu Ray, and entitled “TREATMENT FOR ANGIOGENIC DISORDERS,” the entire disclosure of which is incorporated herein by reference.

### FIELD OF THE INVENTION

The present invention is in the field of pharmaceutical composition, and in particular, in the field of combination therapy for the treatment of disorders associated with aberrant angiogenesis, including ocular angiogenesis and various forms of cancer.

### BACKGROUND OF THE DISCLOSURE

Wet Age Related Macular Degeneration (wet ARMD) is the leading cause of blindness in patients above 55 worldwide. The main targetable biological culprit in this disease state is believed to be the over-expression of Vascular Endothelial Growth Factor (VEGF). VEGF binds to the receptor VEGF-R on the surface of endothelial cells leading to the major observed pathological hallmarks of this disease; namely blood vessel growth, leakage, and hemorrhage under and within the retina, thereby leading to vision loss. The first targeted anti-VEGF therapy ever used in human beings in any setting was Macugen® (pegaptanib sodium), a pegylated covalent conjugate of an oligonucleotide of 28 nucleotides. Macugen® binds to VEGF and disrupts its binding to VEGF-R. However, Macugen® binds to a domain on VEGF that is different than the domain that binds to VEGF-R. Therefore, Macugen® has an allosteric, instead of a direct, effect on the VEGF binding to VEGF-R. Consequently, Macugen® is not a very effective drug. This low efficacy is reflected in the market share of Macugen®, which has gone from 100% to less than 1% after the introduction of other VEGF inhibitors.

The new generation of VEGF inhibitors include drugs such as Avastin® (bevacizumab) and Lucentis® (ranibizumab), both recombinant humanized monoclonal IgG1 antibodies that bind specifically to VEGF, and Eylea® (aflibercept), a recombinant fusion protein having portions of human VEGF receptor 1 and 2 extracellular domains. Eylea® acts as a soluble decoy receptor that binds VEGF, and thereby inhibits the interaction of VEGF with the cognate receptor.

All of the four drugs mentioned above must be injected into the eye on a monthly basis. Although the new generation of VEGF inhibitors are more efficacious than Macugen®, their efficacy is still below the level desired by patients and clinicians. Also, patients become refractory to all of these drugs, continuing with their persistent disease despite the painful and uncomfortable monthly injections.

### SUMMARY OF THE INVENTION

Disclosed are pharmaceutical compositions comprising a therapeutically effective amount of a first compound, wherein the first compound binds the heparin-binding domain of the vascular endothelial growth factor (VEGF); and a therapeutically effective amount of a second compound, wherein the second compound binds to VEGF, thereby inhibiting the binding of VEGF to its cognate receptor. Also disclosed are methods of treating a VEGF-related disorder in a subject, the method comprising identifying a subject in need thereof, and administering to the subject a therapeutically effective amount of a first compound, wherein the first compound binds the heparin-binding domain of the vascular endothelial growth factor (VEGF); and a therapeutically effective amount of a second compound, wherein the second compound binds to VEGF, thereby inhibiting the binding of VEGF to its cognate receptor.

### BRIEF DESCRIPTION OF THE FIGURES

FIG. 1a shows that the results of optical coherence tomography (OCT) studies on Patient 2 (Example 2) treated 33 times with intravitreal ranibizumab demonstrates subretinal fluid (SRF). FIG. 1b shows that the SRF of Patent 2 is resolved after the combination therapy with pegaptanib and bevacizumab.

FIG. 2a shows the fluorescein angiogram of Patient 2 before the combination therapy. Late frames demonstrate leakage from the choroidal neovascular membrane (CNVM) despite aggressive conventional mono-therapy, i.e., 33 times with intravitreal ranibizumab. FIG. 2b shows the results of the angiographic study 6 weeks post combination therapy for Patient 2, demonstrating closure of CNVM with no further leakage.

FIG. 3a shows that the results of optical coherence tomography (OCT) studies on Patient 7 (Example 7) treated 12 times with ranibizumab and 15 times with bevacizumab. The patient demonstrates subretinal fluid (SRF). FIG. 3b shows that the SRF of Patent 7 is resolved after a single combination therapy with pegaptanib and bevacizumab.

### DETAILED DESCRIPTION OF THE EMBODIMENTS

VEGF consists of two independent domains, a receptor-binding domain and a heparin-binding domain (HBD) [Ferrara et al., Nat Med. 2003, 9:669-676.] Pegaptanib binds with high affinity to the heparin-binding domain (HBD) of VEGF. [Lee et al., FEBS Lett. 2008, 582(13): 1835-1839.] This binding causes allosteric conformational changes in the structure of VEGF, which result in poor binding at the receptor-binding domain. Bevacizumab, ranibizumab, and aflibercept, on the other hand, interact directly with the binding of VEGF with its receptors. The present inventor has discovered that pegaptanib interaction with VEGF in conjunction with the interaction of one of the other three compounds causes a synergistic inhibition of the interaction VEGF with VEGF-R.

Thus, in the first aspect, disclosed herein is a composition comprising a first compound and a second compound, where the first compound binds the heparin-binding domain of the vascular endothelial growth factor (VEGF), and the second compound binds to VEGF, thereby inhibiting the binding of VEGF to its cognate receptor.

In some embodiments, the first compound is an aptamer. Aptamers are oligonucleotides that bind to specific target molecules. An aptamer can be either a DNA aptamer or an RNA aptamer, where the sugar in the backbone is either a deoxyribose or a ribose, respectively. In some embodiments, the aptamer comprises a modified ribose, where, for example, one of the hydroxy groups is replaced by another chemical moiety. In some embodiments, the hydroxy group is replaced by a group selected from the group consisting of methoxy, ethoxy, propoxy, fluoro, chloro, and iodo. In certain embodiments, the aptamer comprises at least one non-natural base.

In some embodiments, the first compound is pegaptanib, or a pharmaceutically acceptable salt thereof. Pegaptanib is an aptamer having the following structure.

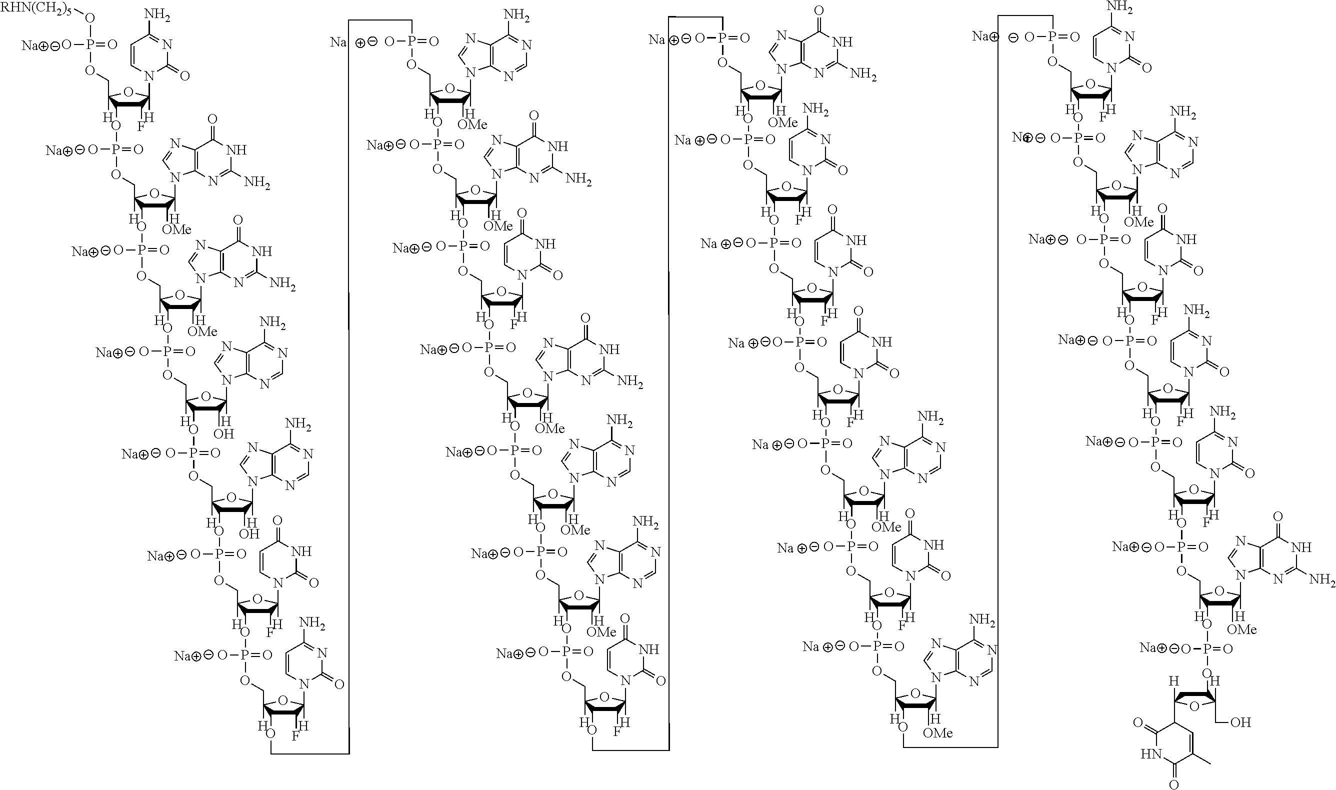

where

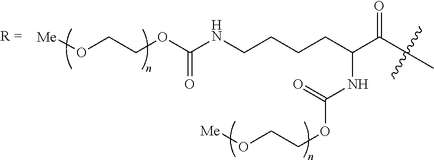

and n is approximately 450.

In certain of these embodiments, the first compound is pegaptanib sodium.

In some embodiments, the second compound binds to a different domain on VEGF than the first compounds. In certain embodiments, the second compound is selected from the group consisting of a polynucleotide, an antibody, a polypeptide, a small organic molecule.

The antibody can be a monoclonal or a polyclonal antibody. In some embodiments, the antibody is a monoclonal antibody.

In some embodiments, the polypeptide is a decoy receptor. In other embodiments, the polypeptide is a small chain polypeptide comprising less than 50 amino acid residues.

In some embodiments, the small organic molecule is an organic compound that comprises two or less amino acids. In certain embodiments, the small organic molecule comprises no amino acids nor any nucleic acids. In some embodiments the small organic molecule has a molecular weight less than 700 g/mol.

In some embodiments, the second compound is selected from the group consisting of bevacizumab, ranibizumab, aflibercept, and a pharmaceutically acceptable salt thereof.

In some embodiments, the first and second compounds are together disposed in the same dosage form. In these embodiments, each administrable dose comprises both the first and second compounds, and an additional excipient, carrier, adjuvant, solvent, or diluent.

In other embodiments, the first and second compounds are not compatible to co-exist within the same dosage form. In some of these embodiments, the first and second compound are packaged separately, but are mixed shortly before administration. In some of these embodiments, the first and second compounds are mixed as the administration is taking place. For example, the first and second compound are placed in separate compartments within the same device and as the composition is administered to the subject, the first and second compound mix, for example, in a passageway leading from the device towards the subject.

Pharmaceutical compositions suitable for use in the method for treating a patient of the present invention include compositions where the active ingredients are contained in an amount effective to achieve its intended purpose. More specifically, a therapeutically effective amount means an amount of compound effective to prevent, alleviate or ameliorate symptoms of disease, or slow, delay, or reverse the progress of disease in the subject being treated. In some embodiments, the overall feeling of well-being in a patient is worse after treatment than before treatment.

The dosage for each compound is determined on a drug-by-drug basis. But in some embodiments, the pharmaceutical compositions disclosed herein comprise between 0.1 to 10 mg of the first compound per administrable dosage form. In some embodiments, the pharmaceutical compositions comprise 0.3, 1, or 3 mg of the first compound per administrable dosage form.

In some embodiments, the pharmaceutical compositions comprise between 0.1-30 mg/kg of bevacizumab, or a pharmaceutically acceptable salt thereof, as the second compound per administrable dosage form. In other embodiments, the compositions comprise between 0.1 to 10 mg of ranibizumab, or a pharmaceutically acceptable salt thereof, as the second compound per administrable dosage form. In yet other embodiments, the compositions comprise between 3-30 mg/kg of aflibercept, or a pharmaceutically acceptable salt thereof, as the second compound per administrable dosage form.

In another aspect, disclosed herein are composition comprising a first compound, a second compound, and a third compound, wherein the first and second compounds are as disclosed above, and the third compound is another compound described as a second compound, above.

In another aspect, disclosed herein are pharmaceutical compositions comprising a first and a second compound, as described above, and a pharmaceutically acceptable carrier, excipient, or diluent.

The term “pharmaceutical composition” refers to a mixture of a compound of the invention with other chemical components, such as diluents, lubricants, bulking agents, disintegrant or carriers. The pharmaceutical composition facilitates administration of the compound to an organism. Multiple techniques of administering a compound exist in the art including, but not limited to, oral, injection, inhalation, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

In certain embodiments, the same substance can act as a carrier, diluent, or excipient, or have any of the two roles, or have all three roles. Thus, a single additive to the pharmaceutical composition can have multiple functions.

The term “physiologically acceptable” defines a carrier or diluent that does not abrogate the biological activity and properties of the compound.

The pharmaceutical compositions disclosed herein can be administered in the formulations prepared for the commercially available pegaptanib, bevacizumab, ranibizumab, aflibercept.

The present inventor has surprisingly discovered that the compositions disclosed herein exert a synergistic effect for the treatment of the pathological angiogenesis. These compositions improve the clinical outcomes exceeding the standards achieved in current medical and clinical practice. The compositions disclosed herein can be used for treatment-naïve patients, as well as for those who have been resistant or become refractory to current therapies or have demonstrated recurrences despite ongoing therapy.

The VEGF molecule has many isoforms with multiple shared and unique domains that interact with designated cell surface receptors and other molecules on cell surfaces, extracellular cellular matrix, and soluble factors. The major signaling for VEGF has been determined to occur through binding to its cell surface receptor, VEGF-R, which itself also has multiple isoforms with distinct activities. These interactions are mediated through residues on exons 3 and 4 of the VEGF molecule via interaction with VEGF-R1 and VEGF-R2 respectively. In contrast, residues within exons 6 and 7 mediate interactions with various heparin-binding motifs as well as interaction with the neuropilin-1 cell surface molecule. This latter molecule has recently been described to play an important role in angiogenesis signaling on endothelial cells through its binding to the VEGF molecule via distinct residues on exon 7 that are separate from moieties involved with binding of VEGF to the VEGF-R. Data suggests that co-signaling of VEGF via NP-1 greatly enhances in a synergistic fashion the signaling generated through binding of the VEGF to the VEGF-R. In various animal and in vitro models, this potentiating activity is thought to amplify the signaling that mediates endothelial cell survival, migration, and proliferation.

The endothelial cells in neovascular vessels both in tumor angiogenesis and pathological ocular angiogenesis “mature,” and as a result become independent of their need for sustained VEGF expression for their ongoing survival. Without being bound to a particular theory, the present inventor has determined that simply blocking VEGF/VEGF-R interaction has diminishing returns with perpetual treatments. The signaling mediated via NP-1/VEGF interaction is also thought to play a role in blood vessel maturation that may indeed make angiogenic vessels independent of their need for further VEGF signaling. This latter effect may be one of the mechanisms by which diseases associated with pathological angiogenesis become resistant or refractory to further VEGF suppressive therapies. In some experimental models, inhibiting signaling through NP-1 provided a dramatic added anti-angiogenic effect when used in combination with anti-VEGF inhibition. Furthermore, inhibiting NP-1 signaling appears to prevent maturation of neovascular tissues, leaving these vessels susceptible to anti-VEGF therapy. As a result of these findings, groups are attempting to develop inhibitors of NP-1 signaling for the treatment of pathological angiogenesis. To date however, these are still in early pre-clinical developmental phases and thus not currently in clinical use.

With respect to the pegaptanib binding site on the VEGF molecule, it is known that the regions within exons 6 and 7 play a role in the heparin-binding activity of VEGF₁₆₅. This domain plays an important role in the sequestration of VEGF within extracellular matrix, and on cell surface heparin sulfate moieties. These sites may serve as reservoirs of potent VEGF activity that are unavailable to neutralization by other anti-VEGF drugs. The binding of pegaptanib within the HBD of VEGF may serve to interfere with VEGF's ability to elude neutralization by VEGF inhibitors. Because pegaptanib seems to be a relatively weak inhibitor of the VEGF-VEGFR interaction especially as compared to bevacizumab, ranibizumab, and aflibercept, it is considered an ineffective stand-alone therapy in wet ARMD. However, it makes more VEGF available for neutralization when administered along with potent anti-VEGF compounds.

Thus, in another aspect, disclosed herein are methods of treating a VEGF-related disorder in a subject, the method comprising: identifying a subject in need thereof, and administering to the subject a therapeutically effective amount of a first compound, as described above, and a therapeutically effective amount of a second compound, as described above.

The term “subject” refers to an animal, preferably a mammal, and most preferably a human, who is the object of treatment, observation or experiment. The mammal may be selected from the group consisting of mice, rats, rabbits, guinea pigs, dogs, cats, sheep, goats, cows, primates, such as monkeys, chimpanzees, and apes, and humans.

In some embodiments the first compound and the second compound are administered more or less simultaneously. In other embodiments the first compound is administered prior to the second compound. In yet other embodiments, the first compound is administered subsequent to the second compound.

A VEGF-related disorder is a disorder in which when the interaction of VEGF, in any of its subforms, with a VEGF receptor, in any of its forms or subforms, is disrupted, then a therapeutic result is obtained. In some embodiments, VEGF-related disorders are those in which a decrease in angiogenesis provides a therapeutic effect to the patient. In some embodiments, the VEGF-related disorder is selected from the group consisting of neovascular age related macular degeneration (ARMD), wet age related macular degeneration (wet-ARMD), diabetic retinopathy, retinal vascular obstruction, ocular tumors, retinopathy of prematurity, colorectal cancer, lung cancer, breast cancer, pancreatic cancer, and prostate cancer.

In some embodiments, the subject is treatment-naïve, i.e., the subject has not been previously treated for the disorder and is being presented to the healthcare provider for the first time. In some of these embodiments, the healthcare provider decides, based on the extent of the progression of the disease or the history of the patient, to treat the patient using the pharmaceutical compositions and the methods disclosed herein. In other embodiments, the patient has been on monotherapy for some time, and the patient has become refractory, i.e., unresponsive or poorly responsive, to the monotherapy treatment. In these embodiments, the healthcare provider decides to treat the patient using the pharmaceutical compositions and the methods disclosed herein in order to improve the clinical outcomes.

In some embodiments, the patient is already on pegaptanib therapy, but the therapy is no longer satisfactory. In these embodiments, the healthcare provider either introduces adjunctive therapy of bevacizumab, ranibizumab, or aflibercept, or the healthcare provider begins treatment with a pharmaceutical composition comprising pegaptanib and one of bevacizumab, ranibizumab, or aflibercept. The patients in these embodiments typically suffer from macular degeneration.

In other embodiments, the patient is already on bevacizumab, ranibizumab, or aflibercept therapy, but the therapy is no longer satisfactory. In these embodiments, the healthcare provider either introduces adjunctive therapy of pegaptanib, or the healthcare provider begins treatment with a pharmaceutical composition comprising pegaptanib and one of bevacizumab, ranibizumab, or aflibercept. The patients in these embodiments typically suffer from macular degeneration or cancer or some other form of VEGF-related disorder.

In some embodiments, the compositions disclosed herein are administered by injection. In certain embodiments, the compositions are injected directly into the diseased organ or tissue. In other embodiments, the compositions are injected intravenously such that the compounds of the composition are distributed systemically. In other embodiments the compositions disclosed herein are administered orally, or in depot.

In another aspect, disclosed herein is a method of re-sensitizing a subject to mono-therapy treatment of a VEGF-related disorder, the method comprising: identifying a subject refractory to the mono-therapy treatment, and administering to the subject a therapeutically effective amount of a first compound, as described above, and a therapeutically effective amount of a second compound, as described above.

In some embodiments, the subject is refractory to antibody therapy, as the therapeutic antibody is described above. In certain embodiments, the subject is treated with a combination therapy, as disclosed herein, at least once. In other embodiments, the subject is treated more than once, for example, twice, three times, four times, or more. Each incident of treatment includes a single administration of the combination drugs in a therapeutically effective amount.

### EXAMPLES

In the following examples, “protocol” treatment means that 0.3 mg of pegaptanib in combination with 0.125 mg of bevacizumab or 0.5 mg of ranibizumab, all in commercially available formulations, were injected intravitreally to a patient, using standard procedures for such injections.

### Example 1

### Case Study of Patient 1

Patient 1 was presented demonstrating lesion activity as measured by active fluid within or under the retina, confirmed by OCT (optical coherence tomography) and fluorescein angiography. Patient 1 had previously received 21 intravitreal injections of either Lucentis® or Avastin®, but had chronic subretinal fluid. The patient provided written consent following a thorough discussion of relative risks and benefits of the potential use of Macugen® in a combination format with simultaneous administration of intravitreal Avastin®.

Macugen® was first injected intravitreally (0.3 mg in 0.09 cc). Optic nerve perfusion and intraocular pressure were assessed. Upon restoration of adequate perfusion and pressure stabilization, the eye was once again sterilized and a second intravitreal injection of Avastin® (1.25 mg in 0.05 cc) was given. Optic nerve perfusion and intraocular pressure were reassessed, and the patient was scheduled for routine follow-up. Following a single administration in this manner, Patient 1 demonstrated complete resolution of subretinal fluid for the first time during the entire course of her therapy.

### Example 2

### Case Study of Patient 2

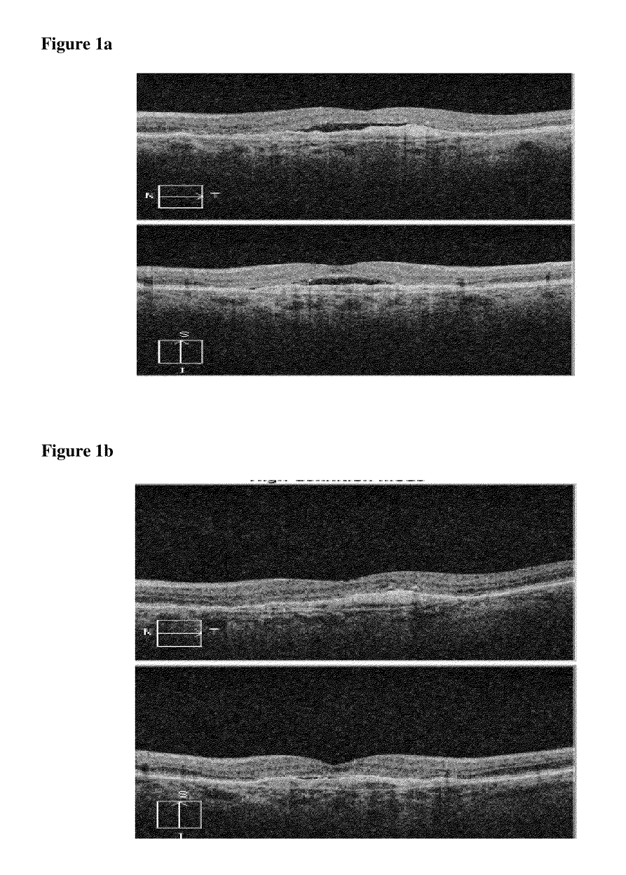

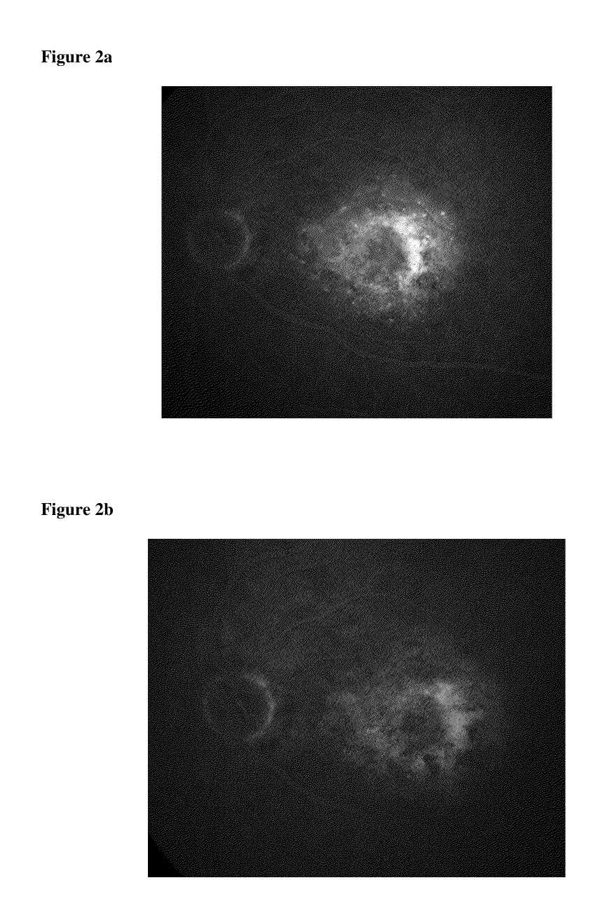

Patient 2 had 33 consecutive treatments with intravitreal ranibizumab with chronic residual subretinal fluid as defined on OCT imaging (FIG. 1a). Following a single simultaneous combination-treatment with pegaptanib and bevacizumab (protocol), there was complete resolution of subretinal fluid and concomitant visual improvement (FIG. 1b). Following that single protocol treatment, Patient 2 who had chronic disease for more than 2 years (while on q4-6 week mono-therapy), remained stable without any fluid recurrence with simple q3 month prophylaxis therapy. This suggests a fundamental change in the underlying disease biology induced by this new treatment protocol. This was confirmed by OCT and fluorescein angiogram results (FIGS. 2a and 2b).

### Example 3

### Case Study of Patient 3

Patient 3 had chronic fluid in the sub-retinal and sub-RPE (retinal pigment epithelium) space following repeated mono-therapy injections of either ranibizumab (11 times) or bevacizumab (3 times). Following a single protocol injection there was resolution of the fluid in the sub-RPE space.

### Example 4

### Case Study of Patient 4

Patient 4 had chronic cystoid macular edema (CME) despite 10 ranibizumab injections. This patient had CME resolution post protocol treatment. Interestingly, this patient had recurrent fluid when either bevacizumab or pegaptanib was used in isolation, validating the rationale that the co-inhibition of both the HBD and RBD within a therapeutic window is effective to achieve maximal efficacy.

### Example 5

### Case Study of Patient 5

A naïve patient, Patient 5, with well defined choroidal neovascular membrane on angiogram showed reduction in lesion size and leakage following single protocol treatment.

### Example 6

### Case Study of Patient 6

Patient 6 received 22 ranibizumab treatments with evidence of persistent lesion activity on both angiogram and OCT. Patient 6 had complete fluid response following one protocol treatment. However, this patient had recurrent fluid following re-administration of bevacizumab mono-therapy (one component of the protocol arm), and then once again had complete resolution following repeat administration of the protocol treatment. This validates the rationale that both drugs co-administered within a therapeutic window help achieve maximal efficacy. Furthermore, once the disease was acutely managed with the protocol treatment, Patient 6 could be managed with ranibizumab mono-therapy alone. This shows that there is a fundamental change in the disease biology induced by the protocol treatment, since this very same patient had been previously recalcitrant to ranibizumab mono-therapy.

### Example 7

### Case Study of Patient 7

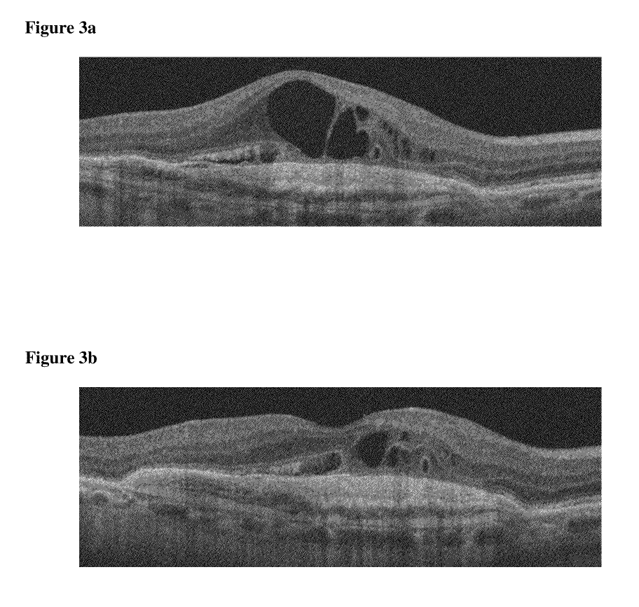

Patient 7 demonstrated chronic subretinal fluid and cystoid degeneration despite photodynamic therapy with verteporfin (X2), monthly ranibizumab (X12), and bevacizumab (X15). Following protocol treatment once, there was dramatic reduction in subretinal fluid and CME with dramatic and immediate visual improvement. Following return to bevacizumab alone, the patient demonstrated relapse of fluid and vision loss, suggesting indeed that it was the combination protocol administration that resulted in the efficacy noted. Following the return to the protocol treatment, Patient 7 obtained his best-recorded vision in 3 years, having regained driving vision for the first time in that period. See FIGS. 3a and 3b.

### Example 8

### Case Study of Patient 8

Patient 8 with poor baseline-vision (20/500) and chronic fluid on OCT as well as leakage on angiogram following 22 mono-therapy treatments, demonstrated progressive improvement in vision, decreased leakage on angiogram, and decreased fluid on OCT following one protocol treatment. When this patient was subsequently returned to mono-therapy, his vision declined again, further validating the rationale of the combination treatment protocol.

### Example 9

### Combination of Pegaptanib and Bevacizumab

A patient with age related macular degeneration is presented. The patient has already received multiple injections with intravitreal bevacizumab but demonstrates either no or incomplete response to therapy as measured by clinical examination and imaging modalities. In addition to the standard bevacizumab therapy, pegaptanib therapy is introduced at a dose according to the ratio of mg compound/kg patient as disclosed herein. The patient shows remarkable improvement.

### Example 10

### Combination of Pegaptanib and Ranibizumab

A patient with age related macular degeneration is presented. The patient has already received multiple injections with intravitreal ranibizumab but demonstrates either no or incomplete response to therapy as measured by clinical examination and imaging modalities. In addition to the standard ranibizumab therapy, pegaptanib therapy is introduced at a dose according to the ratio of mg compound/kg patient as disclosed herein. The patient shows remarkable improvement.

### Example 11

### Combination of Pegaptanib and Aflibercept

A patient with age related macular degeneration is presented. The patient has already received multiple injections with intravitreal aflibercept but demonstrates either no or incomplete response to therapy as measured by clinical examination and imaging modalities. In addition to the standard aflibercept therapy, pegaptanib therapy is introduced at a dose according to the ratio of mg compound/kg patient as disclosed herein. The patient shows remarkable improvement.

### Example 12

### Combinations with Pegaptanib on Naïve Patients

A naive patient is presented with wet ARMD who has evidence of choroidal neovascular membrane. The course of treatment calls for minimizing treatment burden and maximizing vision outcomes. The patient is introduced to a course of treatment using a combination of pegaptanib with bevacizumab, ranibizumab, or aflibercept each at a dose according to the ratio of mg compound/kg patient as disclosed herein. The patient shows remarkable improvement.

### Example 13

### Combination with Pegaptanib in Cancer Therapy

A patient is presented with advanced colorectal cancer who demonstrates further growth in lesion size or disease activity despite multiple administrations of intravenous bevacizumab with or without other chemotherapeutic combinations. In addition to the standard bevacizumab therapy, intravenous pegaptanib therapy is introduced to make the tumor “VEGF-dependent,” and thereby re-sensitized to bevacizumab therapy. The patient shows remarkable improvement.

### Example 14

### Combination with Pegaptanib in Cancer Therapy on Naïve Patients

A naive patient is presented with advanced colorectal cancer. The course of treatment calls for minimizing treatment burden. The patient is introduced to a course of treatment using a combination of pegaptanib with bevacizumab each at a dose according to the ratio of mg compound/kg patient as disclosed herein. The patient shows remarkable improvement.

### Example 15

### Combination of Pegaptanib and Bevacizumab

A patient with age related macular degeneration already on pegaptanib therapy is presented. The patient has become refractory to the treatment. In addition to the standard pegaptanib therapy, bevacizumab therapy is introduced at a dose according to the ratio of mg compound/kg patient as disclosed herein. The patient shows remarkable improvement.

### Example 16

### Combination of Pegaptanib and Ranibizumab

A patient with age related macular degeneration already on pegaptanib therapy is presented. The patient has become refractory to the treatment. In addition to the standard pegaptanib therapy, ranibizumab therapy is introduced at a dose according to the ratio of mg compound/kg patient as disclosed herein. The patient shows remarkable improvement.

### Example 17

### Combination of Pegaptanib and Aflibercept

A patient with age related macular degeneration already on pegaptanib therapy is presented. The patient has become refractory to the treatment. In addition to the standard pegaptanib therapy, aflibercept therapy is introduced at a dose according to the ratio of mg compound/kg patient as disclosed herein. The patient shows remarkable improvement.

What is claimed is:

1. A pharmaceutical composition comprising:
a therapeutically effective amount of a first compound, wherein the first compound binds the heparin-binding domain of the vascular endothelial growth factor (VEGF); and
a therapeutically effective amount of a second compound, wherein the second compound binds to VEGF, thereby inhibiting the binding of VEGF to its cognate receptor.

2. The pharmaceutical composition of claim 1, wherein the first compound is an aptamer.

3. The pharmaceutical composition of claim 1, wherein the first compound is pegaptanib, or a pharmaceutically acceptable salt thereof.

4. The pharmaceutical composition of claim 1, wherein the second compound is selected from the group consisting of bevacizumab, ranibizumab, aflibercept, and a pharmaceutically acceptable salt thereof.

5. The pharmaceutical composition of claim 1, wherein the first and second compounds are together disposed in the same dosage form.

6. The pharmaceutical composition of claim 1, comprising between 0.1 to 10 mg of the first compound per administrable dosage form.

7. The pharmaceutical composition of claim 1, comprising 0.3, 1, or 3 mg of the first compound per administrable dosage form.

8. The pharmaceutical composition of claim 1, wherein the composition comprises between 0.1-30 mg/kg of bevacizumab, or a pharmaceutically acceptable salt thereof, as the second compound per administrable dosage form.

9. The pharmaceutical composition of claim 1, wherein the composition comprises between 0.1 to 10 mg of ranibizumab, or a pharmaceutically acceptable salt thereof, as the second compound per administrable dosage form.

10. The pharmaceutical composition of claim 1, wherein the composition comprises between 3-30 mg/kg of aflibercept, or a pharmaceutically acceptable salt thereof, as the second compound per administrable dosage form.

11. A method of treating a VEGF-related disorder in a subject, the method comprising:
identifying a subject in need thereof, and
administering to the subject a therapeutically effective amount of a first compound, wherein the first compound binds the heparin-binding domain of the vascular endothelial growth factor (VEGF); and a therapeutically effective amount of a second compound, wherein the second compound binds to VEGF, thereby inhibiting the binding of VEGF to its cognate receptor.

12. The method of claim 11, wherein the first compound is an aptamer.

13. The method of claim 11, wherein the first compound is pegaptanib, or a pharmaceutically acceptable salt thereof.

14. The method of claim 11, wherein the second compound is selected from the group consisting of bevacizumab, ranibizumab, aflibercept, and a pharmaceutically acceptable salt thereof.

15. The method of claim 11, wherein the first and second compounds are together disposed in the same dosage form.

16. The method of claim 11, wherein the first compound is administered prior to the second compound.

17. The method of claim 11, wherein the second compound is administered prior to the first compound.

18. The method of claim 11, wherein the first and second compounds are administered substantially simultaneously.

19. The method of claim 11, where in the VEGF-related disorder is selected from the group consisting of neovascular age related macular degeneration (ARMD), wet age related macular degeneration (wet-ARMD), diabetic retinopathy, retinal vascular obstruction, ocular tumors, retinopathy of prematurity, colorectal cancer, lung cancer, breast cancer, pancreatic cancer, and prostate cancer.

20. The method of claim 11, wherein the first and second compounds are administered by injection.
